(19) United States
(12) Patent Application Publication
(10) Pub. No.: US 2010/0087821 A1
(43) Pub. Date: Apr. 8, 2010

(54) FRACTURE FIXATION DEVICE WITH SUPPORT RODS AND SHEATH

(75) Inventors: Roelof Trip, Suwanee, GA (US); James D. Stoneburner, Santa Clara, CA (US); Matthew T. Harmon, Santa Cruz, CA (US); Charles E. Larsen, Tampa, FL (US); Mojan Goshayesh, Atherton, CA (US); Richard A. Hillstead, Suwanee, GA (US); Steven B. Waite, Fernandina Beach, FL (US); Erik H. McDonald, Santa Cruz, CA (US)

Correspondence Address: KNOBBE MARTENS OLSON & BEAR LLP, 2040 MAIN STREET, FOURTEENTH FLOOR, IRVINE, CA, 92614, US

(73) Assignee: NOVALIGN ORTHOPAEDICS, INC., Memphis, TN (US)

(21) Appl. No.: 12/532,611

(22) PCT Filed: Mar. 21, 2008
(86) PCT No.: PCT/US08/57861

§ 371 Date: Sep. 22, 2009

Related U.S. Application Data
(60) Provisional application No. 60/896,352, filed on Mar. 22, 2007.

(51) Int. Cl. A61B 17/56

(52) U.S. Cl. 606/63

(57) ABSTRACT

Fracture fixation devices for placement within the intramedullary canal of a bone are disclosed. The devices disclosed include a tubular support including a liquid impermeable portion and a plurality of elongated members. The devices are typically used with a hardenable cement. Apparatus and methods for removing such devices are also disclosed.

### CROSS REFERENCE TO RELATED APPLICATION

This application claims the benefit of U.S. Provisional Application No. 60/896,352, filed Mar. 22, 2007, the entire contents of which are hereby incorporated by reference.

### BACKGROUND

The present invention relates to orthopedic devices for the surgical treatment of bone fractures and, more particularly, to the fixation and stabilization of fracture sites in long bones with a radially expandable intramedullary device.

Systems and methods for the fixation of bone fractures generally fall into one of three different types: external immobilization of the fracture with casts, splinting devices or external frames; internal fixation of the fracture with plates and screws, and indirect fixation of the fracture by the insertion of an intramedullary device.

The use of an intramedullary device, sometimes called a “nail”, offers several advantages over the other two general methods of fracture fixation. First, the use of an intramedullary nail has the biomechanical advantages of load sharing along the central axis of the bone, torsional and bending stabilization of the fracture both proximal and distal to the fracture site, and resistance to compression and bending forces. Biological advantages include the preservation of the soft tissue envelope at the fracture site, preservation of blood supply to the fracture site, and formation of abundant bone callus around the fracture due to micro-motion of the fragments. Surgical advantages include the need for only small incisions remote from the fracture through the non-traumatized tissues, ease of insertion of the fixation device, and use of the device itself for fracture reduction.

An exemplary intramedullary fracture fixation device is disclosed in International Publication No. WO 2005/112804, which discloses an intramedullary fracture fixation device having an expandable structural frame that is adapted to be placed in the intramedullary canal of a long bone. This system includes a column of surgical fluid, such as bone cement, within which the structural frame acts as a reinforcing cage, and a sheath that is positioned at the fracture site that at least partially surrounds the frame and fluid.

### SUMMARY

The device disclosed herein constitutes an improvement of the intramedullary fracture fixation device, as generally described and disclosed in the above-referenced PCT application. The bone fracture fixation device described herein includes an expandable, generally tubular support that has open proximal and distal ends. The support comprises a plurality of interconnected spaced elongated members and has a liquid impermeable portion.

The liquid impermeable portion of the support is located so that it spans the fracture site of the long bone into which the device is to be inserted. In a first embodiment, the liquid impermeable portion comprises a tubular sheath that is placed over the tubular support. In a second embodiment, the liquid impermeable portion may comprise a coating of a biocompatible material that is integral with the support structure.

The device preferably comprises a wire support, such as a mesh tube, with rods comprising the elongated members. The outer surfaces of the rods are preferably roughened or textured, and more preferably have either a threaded or knurled surface.

In a further aspect of the devices disclosed herein, the liquid impermeable portion comprises at least one flange that extends outwardly therefrom that is adapted to seal against the interior of the intramedullary canal when the device is positioned therein.

Alternatively, in place of the mesh tube, an expandable, stent-like structure or a coiled, spring-like structure may be used for the wire support.

In a further aspect of the invention, the elongated members or rods are secured to the wire support by, e.g., suturing the rods to the wire support, or by interweaving the rods from the outside to the wire support.

In another aspect, the present disclosure relates to an apparatus for removing fracture fixation devices of the type described above from the intramedullary canal. The removal apparatus includes a shaft and a working end wherein the working end includes one or more slots for capturing an end of at least one of said elongated members.

In a further aspect, the present disclosure relates to methods for removing the fracture fixation device from the intramedullary canal by providing the removal apparatus described herein, introducing the apparatus into the intramedullary canal, capturing an end of at least one of the elongated members, manipulating the working end of the apparatus to dislodge the elongated member from the cement and removing the elongated member from the intramedullary canal.

### BRIEF DESCRIPTION OF THE DRAWINGS

FIG. 1 is a schematic view of an intramedullary fracture fixation device described herein shown implanted in the intramedullary canal of a fractured long bone such as a tibia.

FIG. 2 is a fragmentary perspective view of the expandable tubular support of a fracture fixation device described herein comprising elongated rods and a coiled member, the elongated rods being secured to the outer surface of the coil member.

FIG. 3 is a perspective view of an alternative structure to that shown in FIG. 2 in which the elongated rods are carried on the interior of a mesh tube.

FIG. 4 is a fragmentary perspective view of a fracture fixation device described herein similar to that of FIG. 3 and further including a tubular sheath for the liquid impermeable portion, the tubular sheath being carried on the outside of the mesh tube and including upper and lower flanges that seal against the inside surface of the intramedullary canal.

FIG. 5 is a fragmentary perspective view similar of a fracture fixation device similar to that in FIG. 4, except that the sheath includes a plurality of flanges at its upper and lower ends.

FIG. 6 is a fragmentary perspective view of an intramedullary fixation device similar to that shown in FIG. 4, except that the tubular sheath is formed as a coating on the mesh so as to be integral therewith.

FIG. 7 is a perspective view of the tubular support similar to that shown in FIG. 3, except that the elongated rods are threaded.

FIG. 8 is a fragmentary view of a tubular support similar to that of FIG. 7, except that the elongated rods are knurled.

FIG. 9 is a schematic fragmentary view showing the insertion of an intramedullary device as described herein into the intramedullary canal of a long bone, such as a tibia.

FIG. 10 is a schematic perspective view of an implanted intramedullary device described herein that is being subjected to an external energy force that is transmitted to the hardened bone cement through one of the rods in order to soften or break down the bone cement prior to removal of the intramedullary system.

FIG. 11 is a schematic perspective view of an implanted intramedullary device according to the present invention and the working end of an associated tool used during removal of the device.

FIG. 12 is a top view showing the cooperative engagement of the tool of FIG. 11 with the rods of an intramedullary device according to the present invention.

FIGS. 13A, 13B and 14 illustrate a first alternate embodiment to the removal tool of FIGS. 11 and 12.

FIGS. 15 and 16 illustrate a second alternate embodiment to the removal tool of FIGS. 11 and 12.

FIG. 17 illustrates a third alternate embodiment to the removal tool of FIGS. 11 and 12.

FIGS. 18 and 19 illustrate a fourth alternate embodiment to the removal tool of FIGS. 11 and 12.

FIG. 20 is a bar graph showing VAS scores (mean±SD) for both Braid and IM nail groups at different time points and overall scores for both groups; NS indicates no significant difference (p>0.05); means with different characters in each treatment group indicate significant difference among different time points (p<0.05).

FIG. 21 comprises a series of radiographic images demonstrating both Braid (A-E) and IM nail (F-J) treatment at 0, 2 4, 8 and 12 weeks after surgery.

FIG. 22 is a bar graph showing radiographic scores (mean±SD) for both Braid and IM nail systems at different time points, there being no significant difference between the two groups at any time point (P>0.05); means with different characters in each treatment group indicate significant difference among different time points (p<0.05).

### DETAILED DESCRIPTION

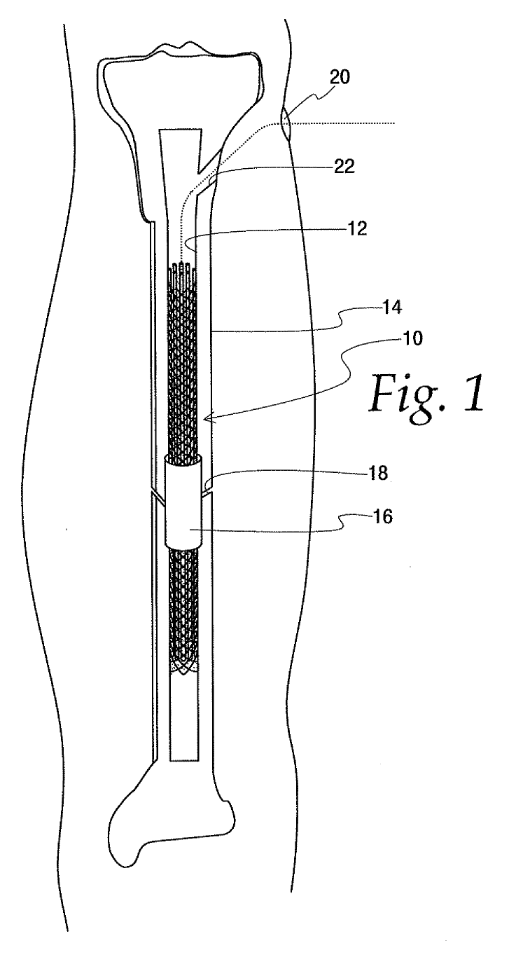

With reference to FIG. 1, an intramedullary fracture fixation device described herein, generally designated 10, is shown in place within the intramedullary canal 12 of a long bone 14, such as a tibia. The device 10 includes a liquid impermeable portion or sheath 16 associated therewith that spans the fracture site 18. The device is introduced into the intramedullary canal 12 through an incision 20 in the skin and an access opening 22 in the cortical wall of the long bone 14 that is preferably oriented obliquely with respect to the center line or axis of the intramedullary canal. Once in place, a bone cement or other hardenable surgical fluid is introduced into the device. The surgical fluid may be contained, at least partially, by the sheath 16 in order to prevent the surgical fluid from seeping into the fracture site 18. Once in place, the surgical fluid hardens and, together with the structural components of the device, provides fixation and stabilization of the fracture site.

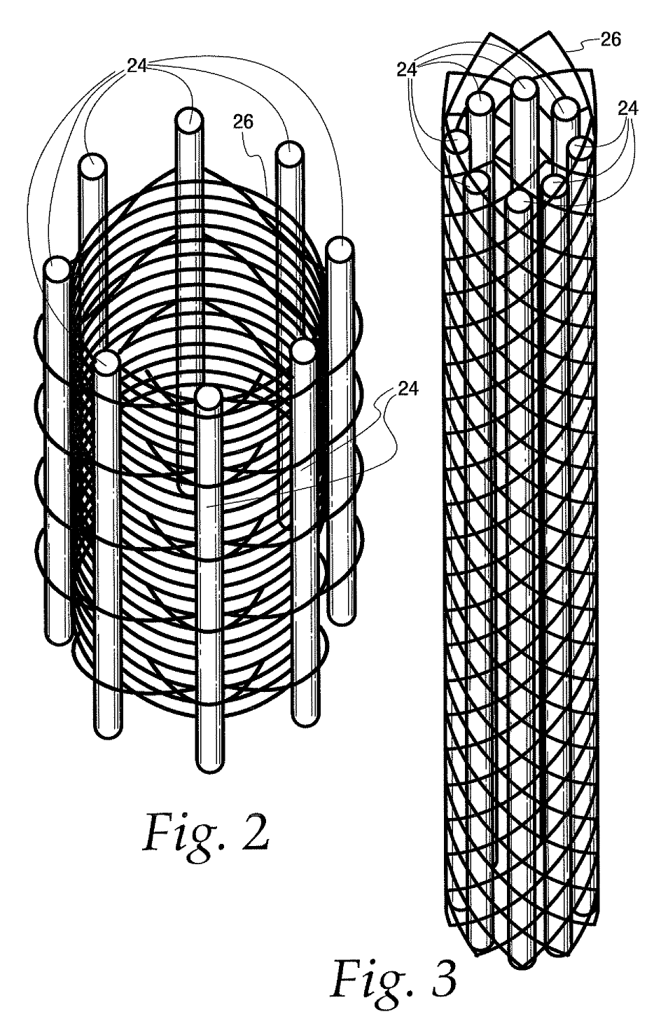

As better seen in FIGS. 2 and 3, the fracture fixation device 10 of the present invention comprises a tubular support made up of a plurality of elongated rods 24 (eight shown, although more or fewer may be utilized) that are associated with a wire-like structure or support 26 that serves to maintain the spatial relationship of the elongated rods 24. The wire support 26 may be made of various biocompatible metals, such as nitinol and titanium. The wire structure 26 is capable of radially expanding or contracting, so as to be radially contracted for introduction into an insertion tube and to radially expand after exiting the tube so as to engage the walls of the intramedullary canal.

The rods 24 are made of a biocompatible material that is sufficiently flexible to allow the rods to bend when introduced into the intramedullary canal through access opening 22. In a preferred embodiment, the rods are made of hardened 455 stainless steel that are 1.6 mm in diameter. Of course, these dimensions are dependent upon the size of the bone with which the assembly is used. As shown in FIG. 2, the elongated rods 24 may be secured to the wire support 26 by interleaving it with the wire support 26 at various locations along the length of the support. Alternatively, the elongated rods 24 may be secured to the wire support 26 by means of sutures, as described in greater detail below.

With reference to FIG. 3, the wire support 26 may form a mesh or braided support. In a preferred embodiment, the mesh support 26 is preferably made up of twenty four super-elastic nitinol wires, each 0.012 inches in diameter. Again, these dimensions are dependent upon the size of the bone with which the assembly is used. The wires are preferably braided in a 1 wire over 2, under 2 pattern, with a pick count of 12 wires per inch. For a preferred system for insertion into the adult tibia, the relaxed (expanded) length of the wire support is approximately 114 mm, with an inside diameter of approximately 8 mm. Alternatively, a stent-like structure may be used in place of the mesh support.

Sutures may be used to secure the elongated rods to the wire support. The sutures are preferably located approximately 2 cm from the ends of the wire support. From rod to rod, the longitudinal position of the suture is preferably varied to spread out the combined tension produced by the sutures on the insertion sleeve into which the system is placed for introduction into the intramedullary canal. The sutures are preferably secured to each rod by a series of overhand knots, with the knots dressed so that they are to the side of the rod and close to the braided support. In practice, the suture is preferably an un-dyed synthetic absorbable suture, such as Monocryl Y415, available from Ethicon. The sutures at one end are moderately tight about the rods, while the sutures at the other end are tightened in a slack loop. This permits the wire support to expand and contract, changing length, while the slack sutures slip along the rods.

The support structure of the present invention is intended to be used in conjunction with a bone cement or other hardenable surgical fluid, such as polymethyl methacrylate (“PMMA”). Bone cement is available from various sources, including Zimmer, Inc., which markets its PMMA bone cement under the trademark OSTEOBOND. Alternatively, a resorbable bone cement may be used, such as a resorbable calcium phosphate. Resorbable cements may be preferred because they are not permanent and do not require removal.

To contain the bone cement when in its liquid state, support 10 is provided with a liquid impermeable sheath 16 on its intermediate portion where the device spans the fracture site 18. The liquid impermeable sheath 16 thus serves to inhibit the liquid bone cement from entering the fracture site 18.

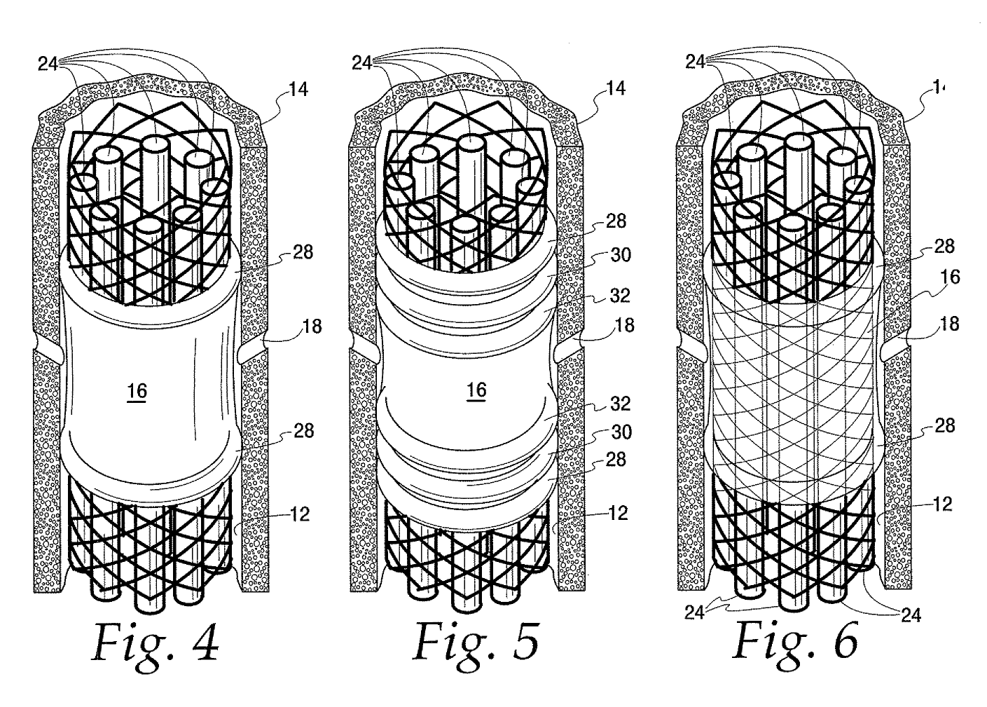

With reference to FIG. 4, the sheath 16 may be formed of a resilient, flexible material, such as a medical-grade silicone, separately from the wire support 26 and elongated rods 24, and then placed thereon. Preferably, the sheath 16 includes a flange 28 extending outwardly therefrom that engages the walls of the intramedullary canal 12. The flange 23 serves to prevent the passage of surgical fluid along the outside surfaces of the sheath 16 toward the fracture site 18. As shown in FIG. 4, the sheath preferably includes both upper and lower flanges 28 so as to more completely isolate the fracture site. With reference to FIG. 5, multiple flanges 28, 30, 32 may be utilized to provide for enhanced sealing with respect to the walls of the intramedullary canal 12. The multiple flanges 28, 30, 32 may be of differing diameters, so as to permit the flanges to seal against different sized intramedullary canals 12.

While the sheaths 16 of FIGS. 4 and 5 are formed separately from the wire support 26/elongated rod 24 structure and subsequently placed thereover, the sheath 16 may alternatively be formed in place on the structure so as to be integral therewith, as shown in FIG. 6, by e.g., coating the mesh tube at the desired location with a medical-grade silicone. The coating is preferably applied in such a way so as to provide proximal and distal flanges 28, like those discussed above in connection with the embodiments of FIGS. 4 and 5. Alternatively, the coating may be applied to provide a sheath 16 of a substantially uniform diameter.

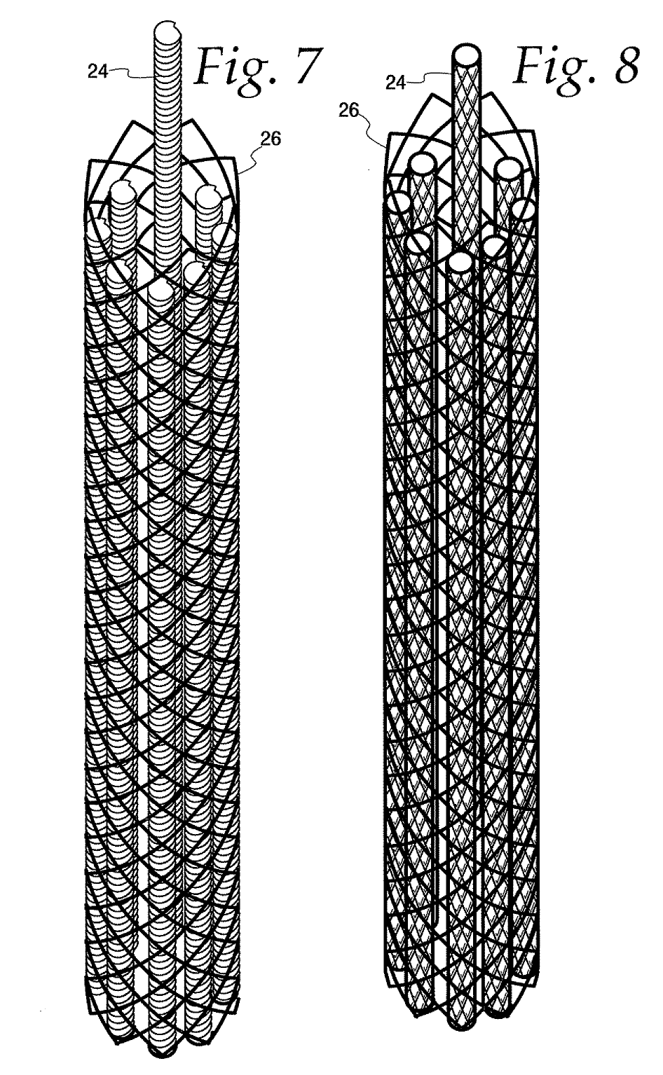

In order to enhance the adhesion of the surgical fluid to the elongated rods, the surfaces of the rods 24 are preferably roughened or textured so as to be provided with a non-uniform surface. As shown in FIG. 7, the surfaces of the rods 24 may be threaded. A particular advantage of the threaded rods 24 is that the threads may assist in the removal of the rods from the hardened surgical fluid in that they may be simply unscrewed therefrom. Further, providing the rods 24 with a threaded surface improves the strength of the hardened implant as well. Alternatively, the surfaces of the rods 24 may be knurled, as shown in FIG. 8, or be provided with other types of textured surfaces.

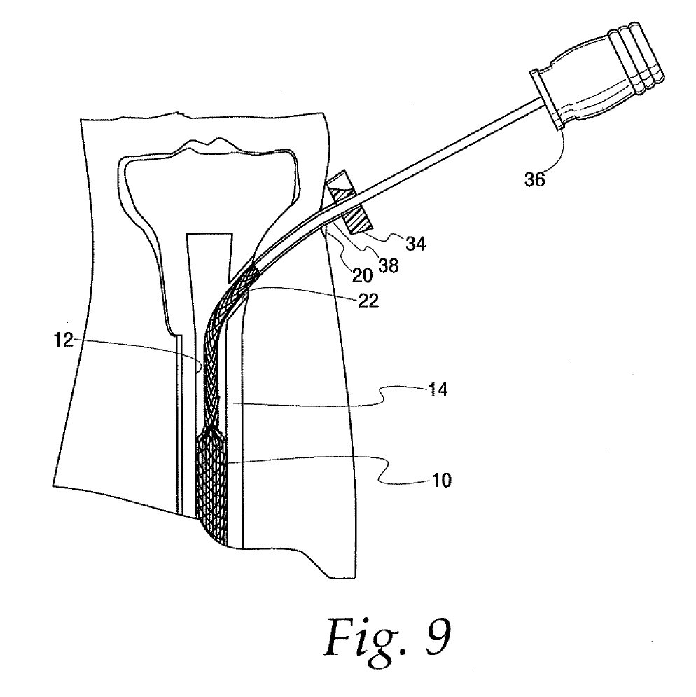

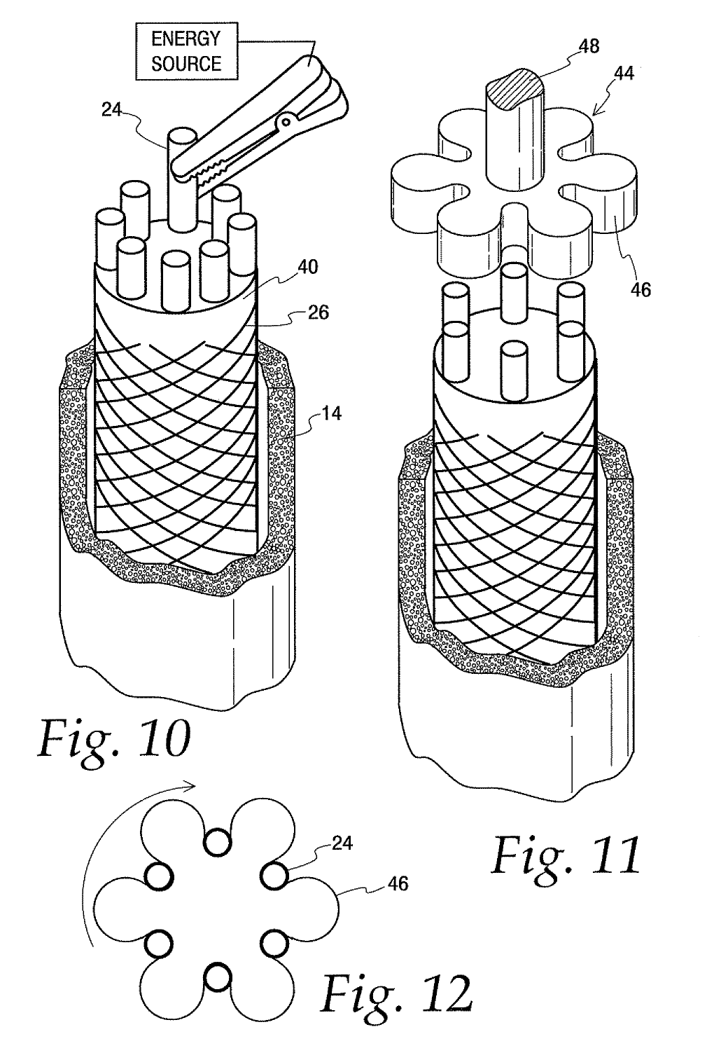

With reference to FIG. 9, the device 10 of the present invention is adapted to be radially contracted so as to fit into the interior of an insertion sleeve or introducer 34 having an O.D. of, e.g., 9.5 mm for percutaneous introduction into the intramedullary canal 12. The device 10 is advanced out of the introducer 34 into the intramedullary canal 12 by means of a plunger 36. The tubing 38 that comprises the introducer 34 is preferably made of a low friction material, such as polytetrafluoroethylene (PTFE). In a typical embodiment, the tubing 38 has an internal diameter of 9 mm and an outer diameter of 9.5 mm. It is intended that the collapsed device 10 be inserted into the introducer tube 38 and the whole system of the device 10, introducer 34 and plunger 36 be packaged and sterilized together. After the device 10 exits the introducer tube 38, it expands radially so as to engage the side walls of the intramedullary canal 12. Then, the surgical fluid is introduced into the interior of the device. The surgical fluid may be introduced through the same portal in the cortical wall as the device. Alternatively, a second portal may be made for the introduction of the surgical fluid. The radial expansion of the wire support and the surgical fluid serve to maintain the device 10 in place in the intramedullary canal 12, so that no further fixation means, such as screws, are contemplated. A study of the use of intramedullary device 10 is reported below.

### Ovine Model Study

### i) Overview

The purpose of this study was to evaluate bone healing of tibial osteotomies treated with an intramedullary construct of the type generally described above composed of a Nitinol wire braid and hardened stainless steel rods (OsteoLign Inc, Duluth, Ga.) followed by injection of polymethylmethacrylate (PMMA) bone cement (“Braid system”) in an ovine model, and compare this technique to a standard interlocking intramedullary (IM) nail fixation.

In the first phase of the study, an in vitro study using ten pairs of sheep tibiae was performed to determine the single cycle torsional properties of the constructs. A middiaphyseal transverse osteotomy was performed in the right tibia of each sheep and then randomly assigned to the Braid system group or IM nail group (n=5/group); the left tibia served as non-treatment intact bone control. In the second phase of the study, twelve sheep were randomly assigned to one of the two treatment groups (n=6/group), Braid system or IM nail group. Following induction of anesthesia and aseptic preparation of the limb, a middiaphyseal transverse osteotomy was performed on the right tibia and the bone repaired with the assigned technique. Following recovery from anesthesia, lameness was evaluated with a visual analogue system (VAS) at 1, 2, 3, 4, 6, 8, and 12 weeks after surgery and bone healing was evaluated radiographically at 2, 4, 8, and 12 weeks after surgery, at which time the animals were euthanatized. After euthanasia, the treated right tibia and untreated intact left tibia were tested in torsion to failure. Bone healing was then evaluated histologically.

For the in vitro study, the maximum torque of the IM nail group was significantly greater than that of Braid system group (P<0.05); whereas there was no significant difference in the torsional stiffness between the two groups. For the in vivo study, the operative time for the Braid system group was significantly shorter than the time for the IM nail group (p<0.05). At 1 and 4 weeks after surgery, VAS scores for the Braid system group were significantly better than those for the IM nail group (P<0.05). At 12 weeks after surgery, there were no significant differences in either maximum torque or torsional stiffness between IM nail and Braid system groups. Radiographic analysis demonstrated that there were no significant differences in radiographic union scores between IM nail and Braid system groups at any time interval. Histologic analysis of the osteotomy at 12 weeks revealed no significant differences in bone healing between the two groups.

Based on the results of the current study, the Braid system for treatment of sheep tibial osteotomy significantly decreased the operative duration time and improved gait at 1 and 4 weeks after surgery compared to standard IM nail fixation. Although the maximum torque for the Braid system was inferior to IM nail fixation at time 0, there was no difference between the two groups with regard to mechanical properties at 12 weeks, radiographic progression of bone union, or histologic evidence of bone healing. The Braid system warrants further investigation to evaluate its potential for use in fracture repair of long bones.

### ii) Materials and Methods:

In vitro study: Ten mature female sheep ranging from 70 to 76 kg (72.5±2.8 kg, mean±SD) with tibial length of 24-27 cm were used in this study. Immediately after euthanasia, ten pairs of tibiae were harvested for analysis of mechanical properties. The surrounding skin, muscle and fascia were removed. The right tibia of each sheep was randomly assigned to the Braid system group or IM nail group (n=5/group), the left tibia served as a non-treatment intact bone control. In the Braid system group, the cranial aspect of the medial tibial plateau was exposed and an 11-mm diameter drill bit was used to access the tibial medullary canal. The canal was then lavaged with lactated Ringer's solution using a pulsatile lavage system (Stryker Corp., Kalamazoo, Mich.), and further cleaned of marrow fat utilizing a long handled bristle brush. The canal was not reamed. A mid-diaphyseal transverse osteotomy then was created using an oscillating saw (Model 1370, Stryker Corp., Kalamazoo, Mich.). The osteotomy was reduced in anatomic alignment with self-locking bone reduction forceps. The Braid system then was inserted into the medullary canal using an insertion tube, which was then pulled back through the medial tibial plateau while holding the cage in place. As the cage emerged from the insertion tube, it expanded to the perimeter of the canal. Approximately 3 cm proximal to the distal end of the tibia, a 5.0 mm hole was drilled medially into the medullary canal. After suctioning the medullary canal of all blood and remaining debris, PMMA cement (Zimmer Osteobond 40/20 kits) was injected in a low-viscosity state with a cement gun through this access hole filling the entire tibial canal in a retrograde fashion until PMMA appeared at the proximal tibial insertion site. After the PMMA hardened, the osteotomy gap was checked to ascertain whether PMMA had leaked into the osteotomy gap. If PMMA was detected in the osteotomy gap, a dental tool was used to remove the PMMA from the gap.

In the IM nail group, the surgical procedure was identical to the Braid system group except that the IM nail (diameter: 8 mm, length: 18.5 cm, Innovative Animal Products, Rochester, Minn.) was inserted in the tibial canal. Two interlocking screws at each end were inserted using a guide device after the tibial osteotomy was reduced.

After completion of the tibial stabilization with either technique, the operated and contralateral non-operated intact tibiae were kept moist with physiological saline soaked towels and biomechanically tested within 6 hours of fixation. The central 10 cm span of the tibiae (5 cm proximal and distal to the osteotomy site) was measured and marked. From the proximal and distal limits of the 10 cm central diaphysis, 6 cm were measured towards the proximal and distal epiphysis. Any bone exceeding these limits was trimmed, yielding a specimen length of 22 cm. Two 4.8 mm holes were drilled at the tibial proximal and distal ends and then the tibiae were potted in polyester/styrene with an alignment jig for testing. Span length for mechanical test was 10 cm. Mechanical testing was performed on a servohydraulic materials testing system (Model 858, MTS Systems Corporation, Eden Prairie, Minn., U.S.A.). The tibiae were tested to failure in torsion in external rotation at 1.5° per second to a maximum of 45° or until failure using a displacement control. Load and deformation data were recorded continuously at 10 Hz with an Analog/Digital board and stored on a read only floppy disk. Stiffness was calculated as the initial slope of the linear portion of each curve. Maximum torque was also obtained from the data for each tibia. To control for individual variance in bone mechanical properties, stiffness and maximum torque were normalized to the contralateral intact tibia and results were expressed as a percent of contralateral control.

Radiography was taken before and after mechanical testing to determine the tibial failure pattern due to mechanical testing.

In vivo study: Twelve mature female sheep ranging from 68 to 78 kg (73.6±5.6 kg, mean±SD) with a tibial length of 24-27 cm were used in this study. The study was approved by the Institutional Animal Use and Care Committee. The right tibia of each sheep was randomly assigned to the Braid system group or IM nail group (n=6/group), the left tibia of each sheep was served as a non-treatment control.

Surgery was carried out under general anesthesia with halothane and oxygen inhalation via endotracheal intubation. Sheep were placed in dorsal recumbency and surgically draped. Using aseptic techniques, a 3-cm incision was made over the medial aspect of the patellar tendon and the cranial portion of the medial tibial plateau external to the joint was exposed and an 11-mm diameter drill was used to access the medullary canal of the tibia. A 7-cm incision was made over the mid-diaphysis of the medial tibial and a mid-diaphyseal transverse osteotomy was made with an oscillating saw (Model 1370, Stryker Corp., Kalamazoo, Mich.). In the Braid system group, another 2-cm incision was made approximately 3 cm proximal to the distal end of medial tibia. A 5.0-mm hole was drilled for later injection of PMMA into the tibial medullary canal. The remaining procedures were identical to the procedure of in vitro study. After the PMMA had hardened following injection, all three incisions were closed in routine fashion. In the IM nail group, the surgical procedure was identical to the in vitro study using fluoroscopic guidance with both incisions closed in routine fashion. The operative time was recorded from skin incision to completion of skin closure for each surgery. After surgery, the sheep were administered with phenylbutazone (500 mg, PO, QD) for pain-relief for 3 days, and allowed to move freely without protection.

At the time of pre-surgery, 1, 2, 3, 4, 8, and 12 weeks after surgery, sheep were evaluated for clinical evidence of lameness with the Visual Analog Scale (VAS) scoring system described by Welsh et al.; Comparison of a visual analogue scale and a numerical rating scale for assessment of lameness, using sheep as a model. Am J Vet Res 1993; 54:976-983.

Radiographs, craniocaudal and mediolateral views, of the tibia were taken immediately after surgery to evaluate osteotomy reduction and pin placement, then at 2, 4, 8 and 12 weeks following surgery. All post-operative radiographs were graded by three independent observers using a scale of 1-4 based on bony union or bridging callus at bone cortices (1=no evidence of callus; union <25%; 2=union 25-50%; 3=union ≧50-75%; 4=union ≧75%) as described by Edwards et al.; Percutaneous injection of recombinant human bone morphogenetic protein-2 in a calcium phosphate paste accelerates healing of a canine tibial osteotomy. J Bone Joint Surg Am 2004; 86:1425-1438.

Sheep were sacrificed at 12 weeks after surgery. After euthanasia, both right and left tibiae were harvested and mechanically tested without removing implants within 6 hours as described in the in vitro study. Torsional stiffness and maximum torque for each specimen were determined. Radiography was also performed before and after mechanical testing to determine the failure pattern resulting from mechanical testing for both right and left tibiae.

After mechanical testing, both right and left tibiae were fixed in 70% ethanol (ETON) solution and a 5-cm section of the mid-diaphysis of the tibia centered over the osteotomy was processed for undecalcified histology as described in Welsh et al.; supra. The tibiae were cut coronally to encompass the region of interest and ground to 100 μm thick sections. The ground bone sections then were stained with a modified Goldner's trichrome method for light microscopic evaluation.

For sample orientation, fine detailed contact microradiographs were taken on a 100 μm ground section of both right and left tibiae with Kodak High Resolution Plates (Kodak, New Haven, Conn.) (18 kVp, exposure time: 2 minutes).

The histologic slides and microradiographs were evaluated by three independent observers according to a qualitative scale (Table 1) that accounted for evidence of bone union and the relative amounts of bone, cartilage, and fibrous connective tissue at the medial and lateral cortices and periosteal callus surrounding the osteotomy site.

TABLE 1
Histologic Scoring System

| | | Score | Score |
|---|---|---|---|
| Region | Histological Appearance | Medial | Lateral |
| Cortical | Gap filled with minimal to no tissue | 0 | 0 |
| Gap | Gap filled predominantly with fibrous | 1 | 1 |
| | tissue | | |
| | Gap filled predominantly with cartilage | 2 | 2 |
| | Gap filled predominantly with woven bone | 3 | 3 |
| | Gap filled predominantly with lamellar | 4 | 4 |
| | bone | | |
| | Total of both sides | 8 | |
| Periosteal | No bridging | 0 | 0 |
| Gap | | | |
| | Predominantly fibrous tissue bridge at | 1 | 1 |
| | osteotomy site | | |
| | Predominantly cartilage bridge at | 2 | 2 |
| | osteotomy site | | |
| | Predominantly woven bone bridge at | 3 | 3 |
| | osteotomy site | | |
| | Periosteal bone predominantly resorbed | 4 | 4 |
| | secondary to cortical union | | |

| | |
|---|---|
| Total of both sides | 8 |
| FINAL SCORE TO THE SAMPLE | 16 |

Analysis of variance (ANOVA) was used to compare lameness scores, operative times, and the in vitro and in vivo stiffness and maximum torque (as % of contralateral control) between groups and for mechanical properties between in vitro time 0 values and in vivo 12 week data. When ANOVA revealed significant differences between treatment groups, a Duncan's Multiple Range Test was performed to separate these differences. A paired Student's t-test was used to compare experimental to contralateral intact bone mechanical properties for each group. Comparison of the radiographic and histologic subjective scores among treatment groups at different time intervals was performed by the Kruskal-Wallis test. A Fisher's Exact test was used to compare osteotomy failure patterns between the two groups. Differences were considered to be significant at a probability level of 95% (P<0.05). All statistical analyses were performed with a commercially available software program (SAS Version 8e, SAS Institute Inc., Cary, N.C.).

iii) Results:

### In Vitro Study

For the in vitro study, maximum torque of the IM nail group was significantly greater than of the Braid system group (P<0.05); whereas there was no significant difference in the torsional stiffness between the two groups (Table 2). For both groups, time 0 mechanical properties were significantly poorer than intact bone strength and stiffness.

TABLE 2
Mechanical Testing Results

| | In Vitro | | | | | |
|---|---|---|---|---|---|---|
| | Braid* | | | IM Nail* | | |
| | Left | Right | | Left | Right | |
| | Control | Experimental | Right/Left % | Control | Experimental | Right/Left % |
| Maximum | 103 ± 26 | 20 ± 4.4 | 21 ± 11^{A} | 72 ± 17 | 25 ± 1.9 | 36 ± 8.0^{B} |
| Torque | | | | | | |
| (Nm) | | | | | | |
| Torsional | 303 ± 26 | 44 ± 6.7 | 15 ± 3.0 | 273 ± 27 | 58 ± 20 | 21 ± 7.6 |
| Stiffness | | | | | | |
| (Nm/radius) | | | | | | |
| | In Vivo | | | | | |
| | Braid^{NS} | | | IM Nail^{NS} | | |
| | Left | Right | | Left | Right | |
| | Control | Experimental | Right/Left % | Control | Experimental | Right/Left % |
| Maximum | 78 ± 14 | 68 ± 20 | 89 ± 28 | 84 ± 12 | 67 ± 29 | 83 ± 38 |
| Torque | | | | | | |
| (Nm) | | | | | | |
| Torsional | 225 ± 33 | 218 ± 41 | 98 ± 16 | 266 ± 49 | 225 ± 70 | 87 ± 30 |
| Stiffness | | | | | | |
| (Nm/radius) | | | | | | |

Mean±SD with different superscript letters (A and B) indicates significant difference in the data as percent of contralateral control between the two treatment groups (p<0.05). The treatment group with a superscript asterisk indicates a significant difference in both maximum torque and torsional stiffness between control and experimental groups. A superscript NS indicates no significant difference in mechanical properties between control and experimental groups.

### In Vivo Study

After surgery, all sheep were able to ambulate without complications. No systemic or local abnormalities occurred during the study. After euthanasia, all tibial osteotomy sites were encompassed by callus.

Operative times for the Braid system group (67.4±11.3 minutes, mean±SD) were significantly shorter than the time for the IM nail group (152±57.1 minutes, mean±SD) (P<0.05).

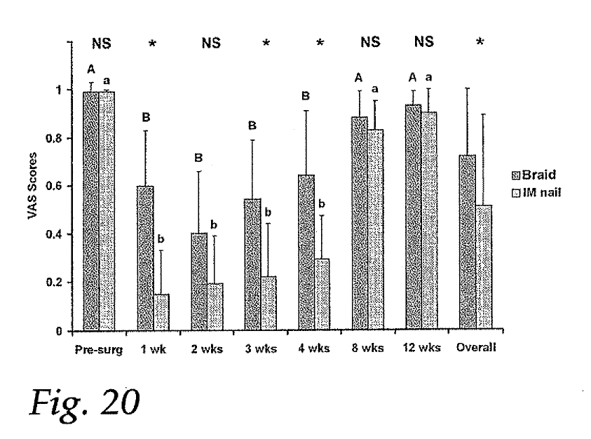

There were no significant differences in pre-surgery VAS scores between the Braid system and IM nail groups. At 1, 3 and 4 weeks after surgery, VAS scores for the Braid system group were significantly better than those for the IM nail group (P<0.05) (FIG. 19). There were no significant differences of the VAS scores between the Braid system and IM nail groups at 2, 8, and 12 weeks after surgery. VAS scores for both Braid system and IM nail groups at pre-surgery, 8 and 12 weeks after surgery were significantly better than those at 1, 2, 3, and 4 weeks after surgery. There were no significant differences in VAS scores for the Braid system or IM nail groups among pre-surgery, 8 and 12 weeks after surgery. Over all times, VAS scores for the Braid system sheep were better than for the interlocking IM nail group.

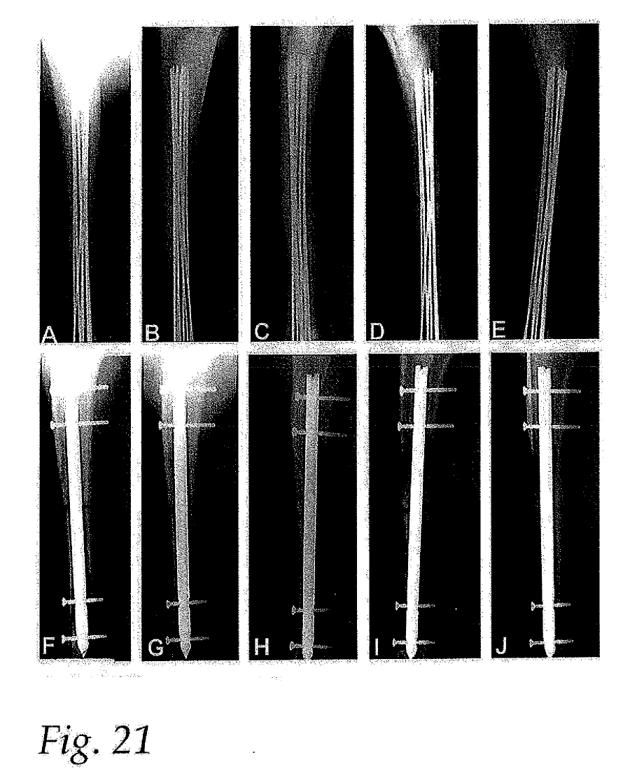

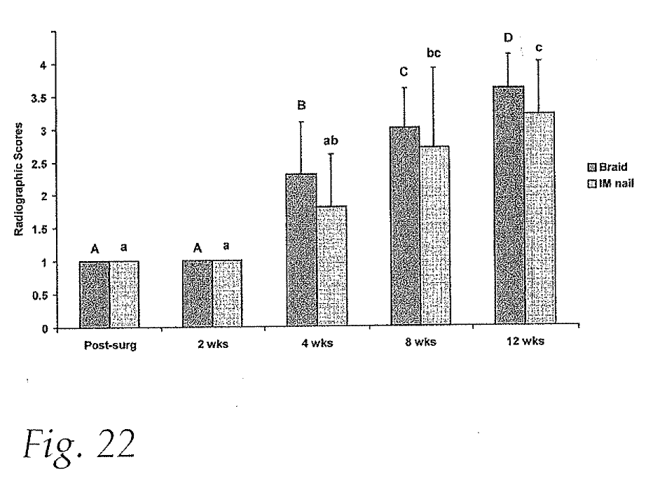

Radiographic analysis demonstrated that there were no significant differences in bone union scores between the IM nail and Braid system groups at any time interval (FIG. 21). In the Braid system group, radiographic bone union scores at 4 weeks post-surgery were significantly higher than those immediately after surgeries and at 2 weeks after surgery (FIG. 22). Radiographic union scores were highest at 12 weeks after surgery compared to those at 4 and 8 weeks after surgery. In the IM nail group, radiographic bone union scores at 8 weeks post-surgery were significantly higher than those immediately after surgery and at 2 weeks after surgery. Radiographic union scores at 12 weeks after surgery was significantly higher than that at 4 weeks after surgery, but not significantly different from scores at 8 weeks after surgery.

For the in vivo study, there were no significant differences in either maximum torque or stiffness between the IM nail and Braid system groups (Table 2). Failure pattern analysis after mechanical testing demonstrated that all contralateral intact bones failed in a spiral pattern, which was the most commonly seen pattern of failure of normal bones under similar testing conditions. Three of 6 bones in the IM nail group failed through the osteotomy sites, whereas only 1 of 6 bones in the Braid system group failed through the osteotomy during torsional testing. There were no significant differences in failure pattern between the two groups (P>0.05).

Histologic analysis demonstrated that there were no significant differences in the cortical and periosteal healing scores between the IM nail and Braid system groups. PMMA cement was rarely detected in the osteotomy site for the tibiae treated with the Braid system.

### Removal

Under certain circumstances, removal of the intramedullary device 10 may be indicated either before or after healing of the fracture. If the bone cement used in the system is not bioabsorbable, then removal of the system 10 will also require removal of the hardened bone cement. Hardened bone cement is usually removed with either a mechanical or ultrasonic reamer, such as those available from Orthosonics Ltd. Before such reamers are used, the reinforcement cage comprising the device 10 must first be removed. Once the rods 24 have been removed, the braided support 26, the sheath 16 and the bone cement may be removed using the reamer.

If the rods 24 are threaded, as described above, then they may be simply unscrewed from the hardened bone cement by means of a drill having a flexible shaft with a specialized socket for attachment to the proximal end of the rods 24. Otherwise, the bone cement will have to be softened or broken down in order to remove the rods therefrom. Hardened bone cement can be broken down by the application of various forms of energy, such as microwave, RF energy, direct electrical current, ultrasound, and/or water jet. Once softened, the tool can be secured to the rods for removal of the rods from the system.

Turning to FIG. 10, an implanted intramedullary device 10 according to the present invention is shown as being subjected to an external energy force in order to break down the hardened bone cement 40. The energy is transmitted to the hardened bone cement 40 through one of the rods 24, as indicated by the attachment of the alligator clip 42 thereto.

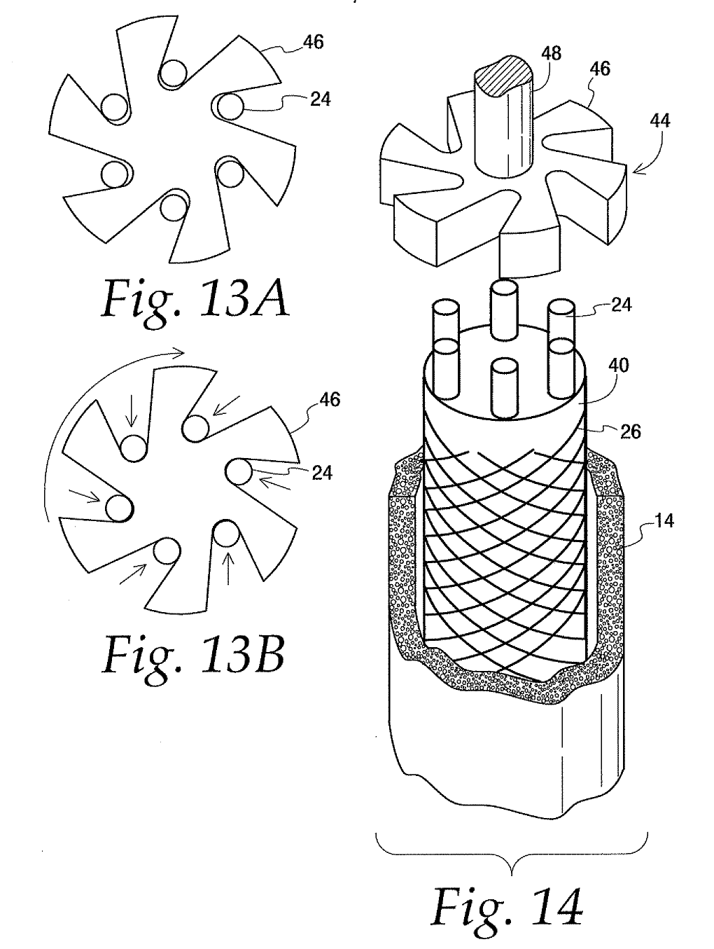

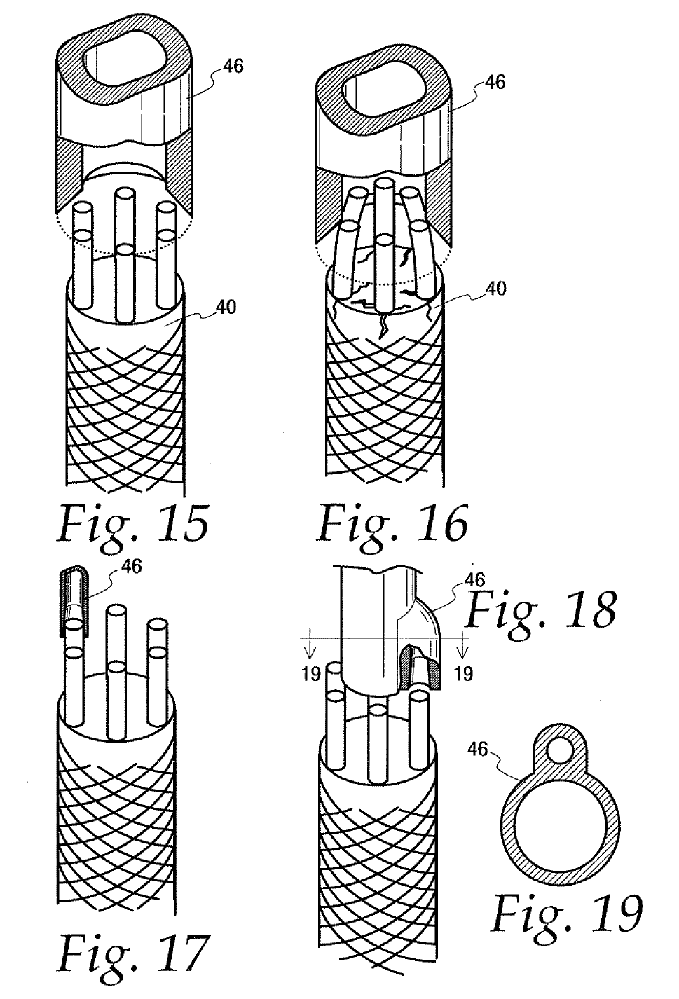

Once the bone cement is softened, a tool 44 is provided that has a working end comprising a driver or socket 46 that is adapted to simultaneously engage the proximal ends of each of the rods 24 in the intramedullary device. As shown, the intramedullary device comprises 6 rods 24 and the socket 46 has 6 slots into which the ends of the rods 24 are received. The socket 46 is preferably secured to a flexible shaft 48, which is rotated by a drill (not shown) in order to break the rods 24 free from the softened bone cement, after which the rods 24 can be extracted one at a time from the intramedullary canal 12. The socket 46 of the removal tool can take different forms, as illustrated in FIGS. 13-19. For example, socket 46 may be star-shaped where the slots are defined by the adjacent prongs of the star-shaped socket. Alternatively, as shown in FIGS. 15-19, socket 46 may be a hollow tubular member whereby one or more rods are captured within the hollow interior of member (socket) 46. Rotation or other manipulation of socket 46 causes rod 24 to break free from the bone cement.

While the invention has been described in terms of certain preferred embodiments, there is no intent to limit the invention to the same. Instead, the invention is to be defined by the scope of the appended claims.

1. A bone fracture fixation device for placement within the intramedullary canal of a fractured bone comprising:
an expandable, generally tubular support having an open proximal end and a distal end, said support comprising a liquid permeable portion and a liquid impermeable portion,
said support further comprising a plurality of spaced elongated members adjoined thereto.

2. The fracture fixation device of claim 1 wherein said liquid impermeable portion comprises a coating of a biocompatible, liquid impermeable material on said support.

3. The fracture fixation device of claim 1 wherein said liquid impermeable portion comprises a tubular sheath placed over said tubular support.

4. The fracture fixation device of claim 1 wherein said tubular support comprises a mesh tube.

5. The fracture fixation device of claim 1 wherein said elongated members comprise rods.

6. The fracture fixation device of claim 5 wherein said rods include an outer surface that is textured.

7. The fracture fixation device of claim 6 wherein at least one of said rods is threaded.

8. The fracture fixation device of claim 6 wherein at least one of said rods is knurled.

9. The fracture fixation device of claim 1 wherein said liquid impermeable portion has at least one flange.

10. The fracture fixation device of claim 9 comprising at least a proximal flange and a distal flange.

11. The fracture fixation device of claim 4 wherein said elongated members are adjoined to said tubular support by sutures.

12. The fracture fixation device of claim 11 wherein said support has a proximal end and a distal end and includes sutures adjoining said elongated members to said tube at said proximal and distal ends.

13. The fracture fixation device of claim 1 wherein said elongated members are sufficiently flexible so as to bend when introduced into the intramedullary canal.

14. The fracture fixation device of claim 1 wherein said elongated members are made of stainless steel.

15. The fracture fixation device of claim 1 wherein said liquid impermeable portion is made of a material that is impermeable to a hardenable biocompatible liquid cement.

16. The fracture fixation device of claim 15 wherein said cement is resorbable.

17. Apparatus for removing a fracture fixation device that includes a hardened cement and a plurality of elongated members held by said hardened cement from the intramedullary canal of a bone, said apparatus comprising:
a shaft and a working end, said working end comprising one or more slots for capturing an end of at least one of said elongated members.

18. The apparatus of claim 17 wherein said working end comprises a star-shaped member including a plurality of prongs defining slots therebetween.

19. The apparatus of claim 17 wherein said working end comprises a tubular member having a hollow interior for capturing said end of at least one of said elongated members.

20. The apparatus of claim 17 wherein said working end is manipulable by said shaft.

21. The apparatus of claim 20 wherein said shaft is rotatable.

22. A method for removing a fracture fixation device from the intramedullary canal of a bone wherein said device comprises a tubular support, said support further including a liquid impermeable portion adjacent to the site of a bone fracture, a plurality of spaced elongated members adjoined to said support and a hardened cement within said support, said method comprising:
providing a removal device including a shaft and working end, said working end including one or more slots for capturing an end of at least one of said elongated members;
introducing said removal device into said intramedullary canal;
capturing an end of at least one of said elongated members;
manipulating said working end to dislodge said at least one elongated member from said cement; and
removing said at least one of said elongated members from said intramedullary canal.

23. The method of claim 22 further comprising removing said cement from said intramedullary canal.

24. The method of claim 23 further comprising softening said cement prior to said removal of said at least one elongated member.

25. The method of claim 24 comprising softening said cement by applying energy to at least one of said elongated members.
